(19) United States
(12) Patent Application Publication
(10) Pub. No.: US 2024/0027451 A1
(43) Pub. Date: Jan. 25, 2024

(54) BIOSENSOR FOR ELECTROCHEMICAL DETECTION OF E.G. MALARIA BIOMARKERS

(71) Applicant: FORSCHUNGSZENTRUM JUELICH GMBH, Juelich (DE)

(72) Inventors: Dirk MAYER, Frechen (DE); Gabriela FIGUEROA MIRANDA, Juelich (DE); Michael PROEMPERS, Juelich (DE); Viviana RINCÓN MONTES, Aachen (DE); Andreas OFFENHAEUSSER, Aachen (DE)

(21) Appl. No.: 18/265,535

(22) PCT Filed: Dec. 7, 2021
(86) PCT No.: PCT/EP2021/084584

§ 371 Date: Jun. 6, 2023

(30) Foreign Application Priority Data
Jan. 11, 2021 (DE) 10 2021 100 290.7

(51) Int. Cl. G01N 33/569; G01N 33/53; G01N 33/543; G01N 27/12

(52) U.S. Cl. CPC G01N 33/56983; G01N 33/56905; G01N 33/5308; G01N 33/5438; G01N 27/125; G01N 2333/165; G01N 2333/445; G01N 2469/10

(57) ABSTRACT

The present invention relates to biomarker sensors with a multielectrode array structure, kits containing them, methods for their production as well as corresponding uses and applications.

The present invention relates to biosensors, in particular based on flexible multielectrode arrays, for the electrochemical detection of biomarkers, preferably biomarkers of infectious diseases, particularly preferably malaria biomarkers, in particular the simultaneous electrochemical detection of multiple biomarkers of the same pathogen or different pathogens, for example malaria biomarkers.

For the detection of malaria, but also for SARS-CoV-2 viruses, various commercially available colorimetric rapid detection tests (RDTs) exist. Detection is based on antibody-antigen interactions, using antibodies as receptor molecules. Unfortunately, such commercial RDTs still lack sufficient analytical sensitivity and the vast majority of these rapid tests do not distinguish between the different malaria parasites or SARS-CoV-2 virus variants. In addition, the receptor antibodies have low thermostability at elevated temperatures. In view of the fact that most regions where malaria is widespread have hot and humid climates, these antibody-based diagnostic systems are of low accuracy. Furthermore, a disadvantage of these receptor antibodies is that they are quite expensive to produce and are sensitive to many chemicals and chemical modifications, which limits their use. In this context, more robust receptor molecules such as aptamers have been proposed. Aptamers are short single-stranded artificial RNA or DNA oligonucleotides, peptides or such molecules with chemical modification. Such aptamers recognise and bind to a target analyte with high specificity and strong affinity. Compared to antibodies, aptamers are thermostable, cheaper and easier to produce and can be easily chemically modified. For this reason, aptamers are an alternative to overcome the difficulties associated with the use of antibody-based tests. Various detection platforms with different converters, ranging from optical to electrochemical systems, have been proposed using aptamers as receptor molecules. These known systems essentially detect protein biomarkers that indicate, for example, the presence of malaria parasites. The protein most targeted is Plasmodium lactate dehydrogenase (PLDH), which is produced by all malaria parasite species. This enzyme can indicate recent infection because its concentration in body fluids correlates with the level of parasitemia. Another common biomarker is P. falciparum histidine-rich protein 2 (HRP-2), which, however, is expressed exclusively by the Plasmodium falciparum parasite. It is present in red blood cells, serum, cerebrospinal fluid and urine from patients infected with this species. Several aptamers have been developed for the detection of these protein biomarkers, such as the 2008s aptamer, which detects the P. falciparum LDH (PfLDH) biomarker (compare, for example, US 2013/0210023 A1; SEQ ID NO. 2). Another aptamer that can detect both PfLDH and P. vivax LDH (PvLDH) proteins is the pL1 aptamer (see for example in US 2012/0316325 A1; SEQ ID NO. 1) described. The so-called LDHp11 aptamer, which is highly selective only for PfLDH, has also been described previously (for example in Frith, K.-A. et al.; Towards development of aptamers that specifically bind to lactate dehydrogenase of Plasmodium falciparum through epitopic targeting. Malaria Journal 2018, 17 (1), 191). An aptamer that selectively detects HRP-2 protein is the 2106s aptamer (see, for example, US 2013/0210023 A1; SEQ ID NO. 7). A combination of these receptor aptamers allows targeted testing of these major malaria biomarkers mentioned above, which is a great advantage in distinguishing between the two more common P. falciparum and P. vivax parasites, and accordingly for the antimalarial treatment indicated in each case.

It has been shown that electrochemical sensors are superior to other detection platforms due to their high sensitivities, ease of use and execution of detection, with the possibility of miniaturising the electrodes, so that such sensors are suitable for point-of-care applications (PoC-applications). The approach based on multi electrode array chips (multi electrode arrays; MEA chips) which has multiple electrodes and combine the advantages of a large detection area and low background noise investigations. Apart from this, MEAs allow the immobilization of different aptamers on different electrodes of the same MEA chip for the simultaneous detection of multiple malaria biomarkers via individually addressable aptasensors. The development of MEAs on flexible substrates appears to be an advantage in the cheap and easy fabrication as well as their point-of-care use as multitarget biosensors.

Important as biomarkers in the study of malaria are in general Plasmodium falciparum L-lactate dehydrogenase (PfLDH), Plasmodium vivax L-lactate dehydrogenase (PvLDH) and histidine-rich protein 2 (HRP-2). PfLDH, PvLDH and HRP-2 can be detected by immunoassays and immuno rapid tests. Such tests usually work colorimetrically, but besides electrochemical and fluorescence-based detection there are other detection methods. In addition, it is known to employ aptamer receptors for the detection of PfLDH, PvLDH and HRP-2.

Nevertheless, the existing detectors still need improvement, especially with regard to their accuracy for the species under investigation, as well as their production.

Another example for the detection of infectious diseases is the detection of coronaviruses, preferably the SARS-CoV-2 virus, especially the joint screening and differentiation of other virus diseases with similar symptoms such as the influenza virus, human respiratory syncytial virus [abbreviation: HRSV] as well as variants of SARS and MERS.

Furthermore, with hundreds of millions of infections and millions of deaths worldwide, the COVID-19 pandemic has demonstrated how inadequate the procedures for detecting, isolating and tracking of outbreaks of emerging pathogens is. The introduction of testing procedures using rapid antigen tests has facilitated rapid qualitative detection of the new SARS-CoV-2 virus and has proven to be an effective measure for the prevention and containment of the spread of the virus. However, it is still time-consuming to detect the virus quantitatively and to distinguish between different virus variants and/or diseases with similar symptoms.

Even more, the lack of digitisation of current diagnostic tests makes them inefficient and hinders an adequate epidemiological surveillance.

Quantitative detections of the virus are necessary because the virus load changes during the course of the infection. Therefore, the detection of the virus load can increase the reliability of tests and avoid false-negative results. The discrimination between different virus variants is currently possible via RT-PCR tests, and the confirmation of a virus infection due to infectious respiratory diseases other than COVID-19 by using different tests, whereby in most cases a biological sample for testing is required in each case. Therefore, the identification of virus variants that may be more infectious and the identification of the correct infectious disease in the person tested is costly and time-consuming. The latter demonstrates a need for a detection platform that can detect and distinguish between the different SARS-CoV-2 variants and multiple pathogens of respiratory diseases to enable differentiated diagnosis of infectious respiratory diseases.

Current testing methods have revealed a bottleneck in the investigation and backtracking of cases of infections, as they only allow analogue tracking of the results. In other words, the assessment of the diagnostic test results and contact backtracking are done manually by doctors or other highly qualified personnel, which complicates and limits testing, backtracking and isolation measures to combat the pandemic. Given the ongoing pandemic and the prediction of future pandemic outbreaks, the simple, rapid, accurate and quantitative identification of emerging pathogens, as well as the possibility to provide digital results for easier epidemiological surveillance, remains a challenge. The discrimination between COVID-19 and other pathogens of respiratory diseases can facilitate the rapid and accurate diagnosis, treatment and isolation of patients and will increasingly gain importance as SARS-CoV-2 becomes endemic in many areas.

Currently, there are no methods or sensor platforms that enable the detection of malaria and Sars-CoV-2, or can be quickly and effectively be adapted to the other.

The object of the present invention was to provide improved sensors for biomarkers, in particular malaria biomarkers, SARS-CoV-2 biomarkers and biomarkers of other respiratory diseases. These sensors should have good thermal and chemical stability, be inexpensive to manufacture, and have high accuracy for the species under investigation.

In addition, an improved detection technology for quantitative multi-target rapid tests for the detection of various infectious respiratory diseases should be provided. Thus, not only the detection of the SARS-CoV-2 virus, but also the joint detection and discrimination of other virus diseases with similar symptoms as COVID-19, e.g., influenza virus, human respiratory syncytial virus, as well as variants of SARS and MERS should be made possible.

Further objects will become apparent to the person skilled in the art when regarding the following description and the claims.

These and further objects, which become apparent to the person skilled in the art when regarding the present description, are solved by the subject matter outlined in the independent claims.

Particularly advantageous and preferred subject matter will be apparent from the dependent claims and the following description.

In the context of the present invention, room temperature means a temperature of 293.15 Kelvin, i.e., 20° C.

Unless further specified, atmospheric pressure (1013 mbarabsolute) and room temperature prevail in all reactions and procedures.

In the context of the present invention, the terms biomarker sensor, multi-target aptasensor, flex-MEA multi-target aptasensor, flex-MEA multi-target apta sensor, flex-MEA chip or MEA chip are used synonymously for the biomarker sensor according to the invention.

The present invention in particular relates to a biomarker sensor having a multielectrode array structure comprising a carrier substrate on which at least two separate electrode sets are arranged and each electrode set comprises one or more electrodes.

Each electrode set is subdivided into, in the following order
i) an incubation zone in which at least one, preferably exactly one, specific aptamer is bound to the one or more electrodes,
ii) a passivation zone in which a passivation layer covers the one or more electrodes,
iii) a contact zone in which the one or more electrodes are configured to be electrically contacted.

An essential feature of these biomarker sensors is that the incubation zones of the individual electrode sets or the incubation zones and a part of up to 95% of the length of the passivation zones of the individual electrode sets are designed to be movable, in each case independently of those of the other electrode sets, and that the contact zones of the individual electrode sets together form a common contact zone.

In this context, the individual, movable parts of the respective electrode sets naturally also comprise the carrier substrate on which they are arranged.

Within the scope of the present invention, the electrodes are not limited in their precise design, but may have conventional, well-known designs. In preferred embodiments, the electrode per se may be a conductor track etched on or from a substrate, or a wire; a conductor path, in particular one produced by lithography, is particularly preferred. The area of the electrode to which the aptamers are bound can have different structures, it is only necessary that the resulting surface of this electrode area is sufficient to bind the desired or necessary amount of aptamer. In preferred embodiments, this part of the electrode, which is also called the aptamer contact area, is comb-shaped, double-comb-shaped or square, particularly preferably square. This (contact) area of the electrode can also be referred to as the aptamer-covered electrode contact or electrode contact for aptamer coverage.

The incubation zones are the area in which the electrodes or electrode sets are immersed in aptamer solutions in order to bind the aptamers to the electrode. In some embodiments, the area includes a little more surface area to leave space between the aptamer solution surface and the chip and to avoid accidentally wetting the chip in places where this is not desired.

In the context of the present invention, the mobility of the individual electrode sets can be achieved in various ways. One possibility is to attach various further parts to a base substrate and to connect them via joints. Another way, preferred according to the present invention, is to manufacture the carrier substrate from a flexible material, whereby the individual electrode sets can then be moved independently of one another due to the flexibility of the substrate.

According to the invention, it is particularly preferred if the carrier substrate is a material selected from the group consisting of polyethylene terephthalate (PET), polyethylene naphthalate (PEN), polydimethylsiloxane (PDMS), polyimide, polyester and agarose, preferably polyethylene terephthalate, or polyethylene naphthalate, particularly preferably polyethylene terephthalate. These materials are characterized, inter alia, by the fact that they are flexible, i.e., bendable, without breaking, and that they are inert to the chemicals used in the manufacture of the biomarker sensors.

Flexible materials within the scope of the present invention can withstand bending, preferably also multiple bending, and bending back to the starting position by 90°, preferably at a radius of curvature of 2 mm to 10 mm, in particular 3 mm to 5 mm, without breaking. This applies in particular to a layer thickness of the flexible material between 30 μm and 1000 μm, preferably 75 μm to 200 μm and particularly preferably between 80 μm and 150 μm, with material thicknesses of 100 μm being highly preferred in individual embodiments.

In further preferred embodiments of the present invention, flexible materials have a lateral elongation (Epsilon—ε) of 0.01 and a bending of 47% at a radius of curvature (r) of 4 mm.

In preferred embodiments of the present invention, the carrier substrate has layer thicknesses between 50 μm and 300 μm, preferably 75 μm to 200 μm and particularly preferably between 80 μm and 150 μm, with material thicknesses of 100 μm being highly preferred in individual embodiments.

A particularly preferred embodiment of the present invention comprises as a carrier substrate a polyethylene terephthalate film having a thickness between 80 μm and 150 μm, in particular 100 μm.

In particular preferred embodiments of the present invention, the biomarker sensor comprises two, three or four, preferably three or four separate electrode sets, preferably with at least two electrodes each, particularly preferably with at least four electrodes each, wherein the electrodes are preferably made of noble metal or carbon, particularly preferably gold or a gold alloy, especially preferably gold.

In further preferred embodiments of the present invention, the aptamers are selected from the group consisting of 2008s aptamer, 2106s aptamer, pL1 aptamer, LDHp11 aptamer and combinations thereof or selected from the group consisting of C5 aptamer, C7 aptamer, C9 aptamer, C11 aptamer, C15 aptamer or I1 aptamer, IH1 aptamer, SG1 aptamer, HCS1 aptamer, NG1 aptamer and combinations thereof (the C7 aptamer binds to the spike protein of the SARS-CoV-2 virus (all variants), C5 aptamer binds to the spike protein of the delta variant, C9 aptamer binds to the spike protein of the alpha variant, I1 aptamer binds to the HA1 protein of the influenza virus, C15 aptamer binds to the spike protein SARS-CoV-2 omicron (B.1.1.529), SG1 aptamer binds to the glycoprotein G of the human respiratory syncytial virus, HCS1 aptamer binds to the HCoV spike protein, NG1 aptamer binds to the glycoprotein G of the Nipah virus).

In view of the ever new mutations of the SARS-CoV-2 virus, it is highly preferred in the context of the present invention to use an aptamer that fits the respective virus variant to be investigated. In this respect, aptamers that bind to spike proteins of mutated SARS-CoV-2 viruses (the exact designations of which are of course not yet known) are also covered by the present invention.

In further preferred embodiments of the present invention, different aptamers or aptamer mixtures are bound in each electrode set, preferably in a first electrode set 2008s aptamer and in a second electrode set 2106s aptamer and in a third electrode set pL1 aptamer and in a fourth electrode set LDHp11 aptamer or in a first electrode set C7 aptamer and in a second electrode set I1 aptamer, or in at least two electrode sets aptamers selected from the group consisting of C5 aptamer, C7 aptamer, C9 aptamer, C11 aptamer, C15 aptamer or I1 aptamer, IH1 aptamer, SG1 aptamer, HCS1 aptamer, NG1 aptamer and combinations thereof.

In particular preferred embodiments of the present invention, the biomarker sensor may additionally be varied in its design with respect to further electrodes. The biomarker sensor may additionally comprise
i) collectively one reference electrode and one counter electrode,
ii) collectively one reference electrode, one counter electrode, and one resistance thermometer,
iii) one reference electrode and one counter electrode per electrode set, or
iv) one reference electrode and one counter electrode per electrode set and collectively one resistance thermometer.

This listing does not imply that the resistance thermometer is the only possibility to be used besides the reference electrode and the counter electrode; it is of course possible to replace this device by others. In preferred embodiments of the present invention, these may be humidity sensors, bending sensors, deformation sensors, pH sensors.

In a preferred embodiment, the present invention relates to multielectrode array chips (flex-MEA chips) manufactured with a flexible carrier substrate.

The material of the carrier substrate is preferably selected from the group consisting of polyethylene terephthalate (PET), polyethylene naphthalate (PEN), polydimethylsiloxane (PDMS), polyimide, polyester and agarose, preferably polyethylene terephthalate or polyethylene naphthalate, more preferably polyethylene terephthalate.

In a preferred embodiment, the multielectrode arrays are structured by wet chemical etching. A stencil passivation process used in preferred embodiments allows partial passivation of the conductive paths on the electrodes at lower cost compared to standard lithography passivation. In preferred embodiments, a low-cost process called “stencil passivation” using parylene C to form the passivation layer (protective layer over the electrode leads) is used to produce the passivation layer. This process costs about half as much compared to a standard lithography passivation.

The electrode arrays of the present invention are divided into different electrode sets, in a preferred embodiment each separated by a free strip. The flexible substrate (polymer) is cut through in a preferred variant according to the present invention, which allows the electrode sub-groups (electrode sets) to be bent independently or each other without separating from (breaking off) the chip.

The embodiment on the basis of a flexible substrate thus allows the incubation of different electrode sets with different aptamer solutions for the simple realisation of the immobilisation of different aptamers for the production of aptasensors with multiple receptors. In this embodiment, the different sets of electrodes can simply be individually bent downwards like fingers and immersed in aptamer solutions.

It is a significant advantage of the present invention that in this way different aptamers can be applied to a sensor with little effort.

The biomarker sensors of the present invention can thus be compared illustratively to a hand, wherein the individual fingers represent the individual electrode sets; due to the mobility of the individual fingers, these can be individually bent and thus individually and independently of each other be immersed in aptamer solutions and incubated with aptamer.

In a preferred embodiment, the electrode sets are modified with different molecules, which may be receptors targeting the same or different analytes.

In a preferred embodiment, it is also possible to apply instead of the desired aptamers other molecules as passivation molecules or receptors without binding affinity to individual or multiple electrode sets. Electrodes modified with these molecules are used as control sensors. This is a subject matter of a preferred embodiment of the present invention.

Further, in a preferred embodiment, the electrode array may comprise reference electrodes, counter electrodes and resistance thermometers.

In a preferred embodiment, the substrate of the individual electrode sets may be tapered on the incubation side to facilitate insertion into an incubation vessel. A chip may comprise two or more electrode sets. Each electrode set may comprise two or more individually electrically addressable electrodes.

The biomarker sensors according to the invention are not limited to the detection of malaria or SARS Cov-2 viruses. Accordingly, in a preferred embodiment of the present invention, the aptamers are not aptamers directed to malaria biomarkers or SARS Cov-2 biomarkers.

Further, it is a subject matter of the present invention to provide a method of preparing biomarker sensors, in particular as described above, comprising the following steps
I) providing a carrier substrate, preferably a flexible carrier substrate,
II) applying a layer of electrode material on the substrate,
III) structuring the electrode material into individual electrodes,
IV) passivation of a part of the electrode material located between the ends,
V) dividing the electrodes into a number of electrode sets,
VI) incubating one end of each electrode set, the aptamer contact area, in aptamer solution.

In preferred embodiments of the present invention, the layer of electrode material is applied on the substrate by means of physical vapor deposition, preferably by means of electron beam assisted physical vapor deposition, particularly preferably after prior application of an adhesion layer.

In preferred embodiments of the present invention, in step III) a photoresist, preferably a positive photoresist, is first applied to the electrode material, from which a mask with the desired patterning of the metal structures is then produced by exposure to light and development. Subsequently, in these embodiments, the unneeded material of the adhesion layer and the electrode layer is removed by means of a wet chemical etching step, and then the photoresist is removed again.

In preferred embodiments of the present invention, the passivation in step IV) is carried out by means of stencil passivation, wherein first a stencil, preferably a polymer stencil, particularly preferably produced using a laser cutting machine, is placed on the surface of the electrodes, and then the conductive leads of the electrodes are partially passivated by means of chemical vapor deposition (CVD), preferably with a parylene C polymer.

In preferred embodiments of the present invention, the subdivision in step V) is performed by cutting the substrate, preferably in such a way that the total number of electrodes is evenly distributed among the resulting electrode sets. Of course, care must be taken in this step that the previously produced electrode structures are not damaged or destroyed by the subdividing/cutting. However, it is also possible to provide a substrate that is already divided accordingly. In the case of polymer-based substrates, this can be done, for example, by producing the polymer in an appropriate form.

Such an already pre-divided carrier substrate represents a preferred embodiment for the biomarker sensors according to the invention and the methods according to the invention.

In connection therewith, there is a further method according to the invention for the determination of biomarkers in body fluids, preferably in blood samples. This method comprises the following steps or consists of the following steps
i) providing a biomarker sensor according to the invention, or a biomarker sensor prepared by the method according to the invention,
ii) applying a body fluid sample to the biomarker sensor,
iii) connecting the biomarker sensor to a measurement device for current, voltage, resistance or frequency,
iv) detecting the measurement signal and outputting the measurement values by the measurement device,
v) analysis of the measurement values, preferably by means of computers, transportable reading device, preferably by means of software and/or calibration data.
vi) optionally storing, transmitting and/or displaying the analysed results.

The statements made in the context of the biomarker sensors according to the invention with respect to the useable materials etc. are also applicable the methods according to the invention. That is, in preferred embodiments, the same materials in the same dimensions can also be used in the methods according to the invention as in the biomarker sensors according to the invention. However, the methods according to the invention are not limited thereto.

Furthermore, in preferred embodiments, the present invention relates to methods for producing biomarker sensors as shown above, preferably flexible gold-multielectrode array chips, as well as a template passivation layer:
a) In preferred embodiments, an electrode-beam assisted physical vapor deposition is used to first deposit adhesion layer on the flexible substrate, which is preferably a polyethylene terephthalate film. This is preferably titanium adhesion layer, in special cases a 5 nm titanium adhesion layer. A layer of a conductive metal is then deposited as an electrode layer via the same process, preferably this is a gold layer, in special cases a 50 nm gold layer.
b) A positive photoresist, which in preferred embodiments of the present invention may be based on novolak resin and naphthoquinone diazide as photoactive substance (an example of this is AZ 5214 E from Microchemicals GmbH, Ulm, Germany), is used in preferred embodiments as a mask for the subsequent patterning of the metal structures and the areas in which the metal layers are to remain are exposed. The coating can then be developed in a developer. In preferred embodiments of the present invention, this may be a developer on the basis of tetramethylammonium hydroxide in H₂O (an example of which is AZ 326 MIF from Microchemicals GmbH, Ulm, Germany) and in some variants may be done for, for example, 70 seconds.
c) A wet chemical etching step is then carried out in preferred embodiments to remove any excess of the deposited electrode layers that do not conform to the desired patterning; in preferred embodiments of the present invention, a potassium iodide-based etching solution may be used (an example of this is TechniEtch AC12, Microchemicals GmbH, Ulm, Germany).
d) The subsequent photoresist removal, to remove the etch mask, is achieved in preferred embodiments by immersing the PET substrate comprising the patterned electrodes in a photoresist remover, preferably based on organic solvent such as acetone, NMP or DMSO, optionally in combination with at least one amine, in particular ethanolamine, especially preferably AZ 100 remover (Microchemicals GmbH, Ulm, Germany), especially preferably in an ultrasonic bath for 10 minutes, followed by rinsing with isopropanol and deionised water.
e) For the partial passivation of the conductive leads, in preferred embodiments of the present invention a procedure called stencil passivation is used. The polymer stencil is preferably made using a laser cutting machine and placed on the surface of the electrodes. The uncovered areas of the conductive leads of the electrodes are then passivated. In preferred embodiments of the present invention, the passivation is carried out by means of chemical vapor deposition (CVD), particularly preferably the passivation is carried out with a parylene C-polymer, epoxy-based (negative) photoresists (such as SU-8 from Microchem Corp.) or (positive) polyimide resists (such as HD 8820 from HD-Microsystems). In some preferred embodiments, a low pressure chemical vapor deposition may be used. In the context of the present invention, passivation therefore means covering or masking a part of the electrode leads which should not be accessible in the finished sensors. These are therefore the parts between the area where the aptamers are to bind and the area where the sensor is connected to measurement devices.
f) In particularly preferred embodiments of the present invention, each flexible polymer chip has a size of 10.5 mm×15.8 mm, with 16 individually addressable electrodes. Each square-shaped electrode with a size of 550 μm×550 μm is arranged on the so-called incubation side of the sensor (the incubation zone), which serves as a base for the flex MEA. In further particularly preferred embodiments of the present invention, each flexible polymer chip has a size of 10.5 mm×24.13 mm, with 16 individually addressable electrodes, in further variants also a size of 10.5 mm×21.5 mm and/or with 20 individually addressable electrodes. Each square-shaped electrode with a size of 550 μm×550 μm is arranged on the so-called incubation side of the sensor (the incubation zone), which serves as the basis for the flex-MEA.
In preferred embodiments, the conductor paths of the individual electrodes can be varied in diameter from the side of their electrical contact to their end to be covered with aptamer, preferably gradually becoming narrower. However, in other preferred embodiments, the dimensions of the chip can also be smaller or larger, depending on the respective application.

Other preferred embodiments of the present invention relate to methods for the determination of a malaria infection and of the percentage of parasitemia in Plasmodium falciparum parasitized blood samples using the biomarker sensors according to the invention. Other preferred embodiments of the present invention relate to methods for the determination of a COVID-19 infection and, if appropriate, concomitantly the virus load in mucus samples, in particular from the mouth and nose, using the biomarker sensors according to the invention, wherein the mucus samples can, if appropriate, be diluted with defined amounts of buffer solution to improve manageability.

A particularly preferred embodiment of the present invention can be described as follows:

In particularly preferred embodiments, the biofunctionalization of the flex-MEA multi-target apta sensor is realised by overnight incubations (16 hours) of the different prepared electrode sets in different previously activated aptamer solutions with concentrations of 0.5 μM for 2008s, pL1, 2106s as well as 0.03 μM for LDHp11.

In preferred embodiments of the present invention, it is possible to carry out an activation of the aptamers, for their immobilisation on the gold electrode surface; this is particularly preferably achieved by their incubation in 10 mM TCEP solution for 1 hour.

This may be followed by multiple washes with Tris buffer and subsequently with deionised water.

In preferred embodiments, this is followed by blocking using 5 mg/ml PEG solution for 7 hours, followed by multiple washes with Tris buffer.

In some preferred embodiments, the multi-target aptasensor is then incubated with lysed Plasmodium falciparum parasite sample for 45 minutes and subsequently washed with Tris buffer.

In preferred embodiments, the electrochemical signal detection is performed with a potentiostat by means of differential pulse voltametry (DPV) measurements, wherein the DPV measurements are performed in particular in redox species solution (5 mM [Fe(CN)₆]^{3-/4-} in 25 mM Tris buffer).

In this context, in preferred embodiments, the obtained mean values of the redox current change after target detection of different electrodes can be evaluated by means of statistical analysis using statistical analysis software. The software that can be used for this purpose is also not limited to a specific one.

Another particularly preferred embodiment of the present invention can be described in exactly the same way as just illustrated, with the difference that C5 aptamers, C7 aptamers, C9 aptamers, C11 aptamers, C15 aptamers and/or I1 aptamers, preferably C7 aptamers and C15 aptamers, in particular C7 aptamers, are used.

The thickness of the gold and titanium metal layers mentioned as a preferred embodiment are not to be understood as limiting. On the contrary, in the context of the present invention these layers may be of any other thickness; depending on the precise application, the layer thicknesses in preferred embodiments of the present invention will be thinner or thicker.

In the context of the present invention, the electrode material is not limited to the gold described as a preferred embodiment; it can be widely varied as long as sufficient conductivity of the electrodes is ensured and sufficient ability to adsorb the aptamers is provided, optionally after prior activation. Examples of other electrode materials preferred according to the invention are carbon, platinum or polymer/metal/carbon composites (polymer here means conductive polymers e.g. PEDOT:PSS (Poly(3,4-EthyleneDiOxyThiophene) PolyStyrene Sulfonate)).

In the context of the present invention, the patterning of the metal structures is also not limited to the wet chemical etching described as a preferred embodiment. Another preferred embodiment is a lift-off process known in the art.

The photoresist removal can be performed with any photoresist removal solution and not only those described as preferred embodiments.

The dimensions of the leads and electrodes may be of any size; they need only be adapted to the precise application situation.

In preferred embodiments, the integration of a reference electrode and a counter electrode in the same chip design may also be included.

The resistance thermometer (RTD) used in preferred embodiments is not limited to a specific design, size or resistance value.

The stencil fabricated for the passivation may be of any material and of any shape covering the intended passivation area; its fabrication is not limited to the use of a laser cutting machine described as a preferred embodiment.

Moreover, the stencil passivation described as a preferred embodiment is not the only method of passivating the conductive leads. In other preferred embodiments of the present invention, other polymers or insulating materials may be used as passivation layers. Examples of preferred materials include polyimides, epoxy-based negative photoresists (such as SU-8 from Microchem Corp.).

In preferred embodiments of the present invention, the aptamer receptors are selected such that they bind to different epitopes of the same biomarker and/or to other biomarkers which are indicative of the same disease and/or to an agent that interferes with the binding to the biomarkers (possibly causing false positive results).

In further preferred embodiments of the present invention, the outputs of the sensors treated with different receptors are analysed by combining them with logic gates, allowing for unambiguous test results. In still further preferred embodiments of the present invention, the outputs from different sensors treated with the same receptor are averaged to increase redundancy.

In the context of the present invention, the method described above as preferred, in which different prepared electrode sets are incubated with the different aptamer solutions, is not the only way to immobilise different aptamers. This can be achieved in other preferred embodiments by many other methods, such as in particular electrochemical molecular stripping, printing, spotting or lithography. The aptamers may be anchored to the electrode via monothiol bonds or via multithiol bonds or via other covalent bonds.

The specific concentrations and specific aptamers mentioned as particularly preferred for the preparation of the multi-target aptasensors of the present invention can be extended in other variant of the present invention to any concentration and any different type of aptamer receptors targeting any other biomarker. In addition, the incubation time for aptamer immobilisation can be varied. The multi-target aptasensor of the present invention can be applied to the immobilisation of at least two different aptamer receptors and is not limited to a maximum number of receptor molecules.

In further preferred embodiments of the present invention, aptamer receptors (detection aptamers) modified with catalytically active moieties (specific for the corresponding analyte) may be added to the analyte sample to increase the electrochemical signal. In still further preferred embodiments of the present invention, these catalytically active species of the detection aptamers may be enzymes, preferably horseradish peroxidase, or nanoparticles, preferably platinum nanoparticles.

In further preferred embodiments of the present invention, conductive polymers such as poly(3,4-ethylenedioxythiophene) polystyrene sulfonate may be used as an aptamer-embedding electrode coating to increase the density of aptamers on the surface of the electrodes. In further preferred embodiments of the present invention, conductive 3-D scaffolds, i.e. micro- and nanoscaffolds made of metals, carbon and conductive polymers, may be used for this purpose.

In further preferred embodiments of the present invention, instead of the aptamer activation by TCEP described as preferred, the aptamers may be anchored to the electrode by other means, particularly preferably by oligonucleotides such as poly A or with other sequence, by diazonium coupling reaction, by EDC:NHS or similar coupling reaction, (3+2)-cycloaddition between alkyne and azide groups or another coupling reaction based on covalent or electrostatic bonds.

In further preferred embodiments of the present invention, as rinsing medium or washing solvent helix water, tap water, drinking water or any other solvent capable of removing the excess of molecules/biological solutions can be used.

In further preferred embodiments of the present invention, other substances may be used in place of the PEG shown above in a preferred variant to prevent non-specific binding to the electrode. In preferred embodiments of the present invention, these may be alkanethiols of different termination and length, BSA (bovine serum albumin), HSA (human serum albumin) or the like, betaines or oligosaccharides of different termination, side chains and lengths.

In further preferred embodiments of the present invention, the incubation time of the sensor with the analyte sample can be widely varied and depends on the specific surrounding conditions of the exact individual case, in particular also depending on the binding constant of the binding of the aptamers to the corresponding analyte (and consequently also depending on the temperature, for example).

In further preferred embodiments of the present invention, microfluidic channels can be integrated on the surface of the electrodes to reduce the detection time of the target molecules.

In further preferred embodiments of the present invention for obtaining electrical detection signals, besides potentiostats, in principle any suitable device for generating and measuring any electrical/electrochemical signal (current, potential, frequency, etc.) can be coupled to the biomarker sensor according to the invention. Accordingly, in preferred embodiments of the present invention, these are transportable reading devices, Bluetooth and/or wireless transmitters.

In further preferred embodiments of the present invention, a counter electrode and a reference electrode may be associated with individual electrodes. In these embodiments, the aptamer-covered electrode contacts/electrode contact for aptamer covering are in preferred embodiments comb-shaped, particularly preferably double comb-shaped, and the counter electrode and the reference electrode can enclose the aptamer-covered area (area to be covered with aptamer) of the measuring electrode.

Furthermore, the sensor chip of the present invention may also comprise microfluidic channels by means of which the respective sample may be directed to the electrodes, thereby reducing the amount of sample required and reducing the detection time. Such microfluidic channels are preferably made of a polymer, particularly preferably a polymer having a water contact angle of less than 90°.

In a similar manner, other aptamers that are biomarkers of different SARS-CoV-2 variants or of different respiratory viruses that cause symptoms similar to COVID-19 can be immobilised to produce a multitarget flexMEA biosensor for infectious respiratory viruses. Biomarkers of other viruses include the haemagglutinin (HA1) protein of the influenza virus, the glycoprotein G of the HRS virus and the spike protein of the MERS-CoV (middle east respiratory syndrome-related coronavirus).

Within the scope of the present invention, virtually any disease-specific biomarker can be detected based on the principle of the invention, it is only necessary that the necessary aptamers are known or found and can adhere to electrodes in the sense of the present invention.

Further subject matter of the present invention are biomarker sensor kits comprising or consisting of at least one biomarker sensor according to the present invention without aptamers already being bound, a preparation comprising at least one biomarker-specific aptamer, as well as optionally further analysis material, preferably pipettes, biosensor chips, transportable reading devices, chemicals, preparation tubes (Eppendorf tubes) and the like.

In connection with the term “kit”, it should be noted that this kit does not necessarily have to be suitable for the “trouser pocket”. Rather, it is intended to express that prior to analysis, both the biomarker sensor as described above as well as the preparation comprising the biomarker-specific aptamer are present separately from each other, although they are matched and belong to each other. In addition, the kit is also intended to be able to comprise multiple detection sensors and multiple aptamer preparations also designed for different biomarkers.

Although in the method for determination of disease-specific biomarkers according to the present invention, the body fluid is not limited to a specific body fluid, it is self-evident for the person skilled in the art to select the body fluid based on whether the disease-specific biomarkers he is looking for are present in the particular body fluid.

In preferred embodiments of the present invention, particularly when testing for malaria, the body fluid is blood samples.

In other preferred embodiments of the present invention, in particular when it shall be tested for respiratory infections such as COVID-19, influenza or MERS, the body fluid is a mucus sample, in particular from the nose and/or throat.

The body fluids may be processed or prepared prior to use in the method of the present invention according to procedures commonly used in the art.

It should be noted that the method of the present invention is particularly suitable and intended for examining body fluid samples that are already available as such, i.e., the collection of body fluids is not part of the method of the present invention.

In the same way, the biosensor according to the invention can be implemented as a technology platform for the detection of many different infectious diseases caused by viruses, bacteria, fungi or parasites. But also neurodegenerative diseases (detection of misfolded proteins) and chronic diseases (diabetes mellitus, cardiovascular diseases) can be detected or monitored.

In this respect, the present invention can serve as a technological platform with which seasonal infectious diseases and treatment efficacy can be monitored, and has the potential to enable epidemiological surveillance due to the data transfer capabilities.

Therefore, the present invention may also serve as an enabling technology for early application to emerging diseases and the prevention of future pandemics.

Also subject matter of the present invention are uses of the biomarker sensors according to the invention or of the biomarker sensors produced by the method according to the invention, or of the biomarker sensor kits according to the invention for qualitative or quantitative determination of disease-specific biomarkers, preferably malaria biomarkers, particularly preferably in body fluid samples, especially preferably in blood samples or mucus samples, the latter preferably originating from the nose or throat.

Furthermore, the biomarker sensors according to the invention are used as substrates in automated multititer plates.

In particular encompassed by the present invention are uses of the biosensors according to the invention, the biomarker sensors produced according to the invention, or the biomarker sensor kits according to the invention for the determination of malaria biomarkers or SARS-CoV-2 biomarkers, particularly preferably in body fluid samples, especially preferably in blood samples or mucus samples, the latter in particular from the throat and/or nose, most preferably as a substrate in automated multititer plates or in PoC devices.

Accordingly, the biosensor according to the invention can also be used in particular in the context of point-of-care applications or PoC diagnostic tests, for the selective and sensitive detection of infectious respiratory diseases, whereby the present invention can be determined the quantification of the virus load of the infection, if one is detected. The device can be easily operated by anyone, for example at home, at school, in hospitals and so on. In this use case, the device comprises a disposable chip that will be impregnated with a mucus sample from a patient's nose or throat and a reusable portable reading device, similar to that known from PoC glucose sensors. The reading device can process the data from various tests and transmit the data, for example via WiFi or Bluetooth, but also wired, for example to a smartphone, to the treating physician, or to a central health facility.

In further preferred embodiments of the present invention, after binding of the biomarker to the sensor surface, additional biomarker-specific aptamers modified with catalytically active groups and amplifying the electrochemical signal can be added to the analyte.

The sensors of the present invention are less sensitive to environmental influences such as temperature, chemicals, and storage time compared to antibody sensors, and are also less expensive to manufacture. Nevertheless, with the biosensors of the present invention, the evaluations of the tests can be performed digitally, qualitatively and quantitatively. With the sensors of the present invention, it is possible to analyse multiple biomarkers simultaneously, in particular PfLDH-, PvLDH- and HRP-2, and Plasmodium parasite species can be distinguished.

An advantage of the present invention is that by combining the signals from multiple electrode sets for the detection of different biomarkers, the discrimination of the parasite species is made possible and the redundancy of infection detection is increased.

Another advantage of the present invention is that chips with small dimensions can be manufactured, and these small dimensions of the chip and the electrodes enable the analysis of small volumes of blood samples. In addition, it is advantageous that the sample volume can be further reduced by using microfluidic channels.

Furthermore, it is an advantage of the present invention that, in contrast to previous diagnostic tests, digitisation is quite easily possible and thus by the present invention an (appropriate) epidemiological monitoring of/by the determined sensor data is made possible. This is because the electronic connection of the sensors and the electronic gathering of the measurement data the latter can easily be processed and handled.

In the following, the invention will be explained in more detail with reference to the figures. The figures are not necessarily to scale and are simplified. Thus, usual measures etc. familiar to the person skilled in the art are not necessarily shown (interconnection, walls, exact molecular structure etc.) in order to facilitate the readability of the figures.

### FIGURE DESCRIPTION

FIG. 1a) is a top view of a biomarker sensor/flex MEA layout 1 according to the present invention. The double dashed lines 4 represent the cutting strips 3 along which the subdivision of the originally non-subdivided workpiece took place by means of cutting. These cutting strips 4 divide the electrode array into different electrode sets ES-1 to ES-4, facilitating the immobilisation of different aptamers per electrode set. The incubation side/incubation zone I, passivation area/passivation zone P and contact field area/contact zone K are also indicated. The incubation zone I is the area with which the various electrode sets ES-1 to ES-4 are incubated in the respective aptamer solutions in order to fix the aptamers on the electrode contacts 3. For this purpose, the different electrode sets ES-1 to ES-4 can be bent independently of each other, due to the flexible substrate, in order to incubate them individually. Also shown in the figure is the passivation zone P, in which the conductive paths of the respective electrodes are covered and protected by a passivation layer, preferably of parylene C polymer. In the contact zone K, the respective electrodes or the sensor are electrically contacted and connected to measuring devices, such as preferably potentiostats.

FIG. 1b) is a side view of the flex-MEA chip 1 shown in FIG. 1a), in which the preferred materials are indicated; for the substrate PET, for the electrodes gold and for the passivation layer parylene C-polymer. As can be seen from this figure, the electrode extends from the incubation zone I via the passivation zone P to the contact zone K. In the area of the incubation zone I and the contact zone K, the electrode is not provided with a passivation layer, so that a covering with aptamers or a contacting remains possible. Only in the passivation zone is the electrode covered with the passivation layer for protection.

FIG. 2a) is a top view of a biomarker sensor 1, as also shown in FIG. 1a). In addition, however, individual counter electrodes 5 and reference electrodes 6 are now shown for each electrode set ES-1 to ES-4; each set of working electrodes (i.e. each electrode set ES-1 to ES-4) therefore has an associated counter electrode 5 and an associated reference electrode 6. In addition, a resistance thermometer 7 is present in the passivation zone P, the contacts of which are guided into/across the contact zone K. The double dotted lines also indicate here the cutting area 4 for the making of different electrode sets ES1 to ES-4; here, in contrast to FIG. 1, it is still indicated here that the individual cuts for subdividing the substrate and separating the individual electrode sets ES-1 to ES-4 can be made as far as the passivation zone P.

FIG. 2b) is a top view of a biomarker sensor 1 as illustrated in FIG. 2a), with the difference that the electrode sets ES-1 to ES-4 do not have individual counter electrodes 5 and reference electrodes 6. Here, all four electrode sets ES-1 to ES-4 share a counter electrode 5 and a reference electrode 6.

FIG. 2c) is a schematic view of a biomarker sensor 1 as illustrated in FIG. 2a), with the difference that only three electrode sets ES-1 to ES-4 are shown here. Also shown exemplarily in this figure are alignment marks that can be used for orientation if several lithography steps have to be carried out. These are equally applicable to the variants of the other figures, but are not illustrated there for simplicity; on the other hand, they are shown in this figure, but are not absolutely necessary.

FIG. 3 shows the graphical plot of the detection data of the specific biomarkers in samples of Plasmodium falciparum parasite blood samples of the respective specific aptasensors. a) 2008s aptamer, b) pL1 aptamer, c) LDHp11 aptamer and d) 2106s aptamer. The control represents the signal measured with uninfected blood (uRBC). The percentages represent the percentage of parasitemia. The dashed line indicates the threshold and the ordinate shows the relative peak current change per area. It is evident from this figure that the higher the parasitemia, the higher the measurement signal and thus the biomarker sensors according to the present invention can be used not only to qualitatively detect the presence of the biomarkers corresponding to the aptamers, but also to quantitatively detect their quantity.

FIG. 4 shows a different embodiment of an electrode arrangement. In the previous figures, the aptamer-covered electrode contacts/electrode contact for aptamer covering 3 were each shown as squares. Here, the electrode contact for aptamer covering 3 is designed in a double comb-shaped arrangement. In this embodiment, a single electrode is shown, which is arranged together with an enclosing counter electrode 5 and an enclosing reference electrode 6. Such an arrangement can be used as an alternative to the electrode arrangements shown in FIGS. 1 and 2. Furthermore, some size indications are given in this figure by way of example. However, these are not limiting, but only exemplary and represent a manufactured embodiment based on inch dimensions (the distance between the individual comb rods in the example shown is 0.05 mm and is not shown in the figure for the sake of better legibility); it is possible to deviate significantly from the dimensions given, in particular the size ratios in embodiments can be arranged diverging from by between 80% and 500%, relative to the sizes given.

FIG. 5 is a reproduction of the biomarker sensor shown in FIG. 1a), with the difference that in FIG. 5 a specific embodiment with specific size and length specifications is shown, as it was also carried out.

FIG. 6a is a reproduction of the biomarker sensor shown in FIG. 2a), with the difference that in FIG. 6a) a specific embodiment with specific size and length specifications is shown, as it was also carried out.

FIG. 6b is a reproduction of the biomarker sensor shown in FIG. 2b), with the difference that in FIG. 6b) a specific embodiment with specific size and length specifications is shown, as it was also carried out. The inset shows the meander structure of a resistance thermometer (resistance temperature detector, RTD) 7 with specific dimensions as used in this specific embodiment.

FIG. 6c is a reproduction of the biomarker sensor shown in FIG. 2c, with the difference that FIG. 6c shows a specific embodiment with specific size and length specifications, as it was also carried out.

FIG. 7 shows the graphical plot of the sensor signal against the concentration of the biomarker (S protein) of the SARS-CoV-2 virus. The sensor signal was measured with a flex-MEA modified with the C7 aptamer. The specific detection was performed in the sample medium in a concentration range from 1 fg/ml to 100 ng/ml. The dashed line indicates the threshold value, the abscissa the concentration of protein in the sample and the ordinate shows the relative peak current change per area. It is evident from this figure that as the concentration increases, the measurement signal is higher and thus with the biomarker sensors according to the present invention not only qualitatively the presence of the specific biomarker (S protein) of the SARS-CoV-2 virus can be detected, but also quantitatively its amount.

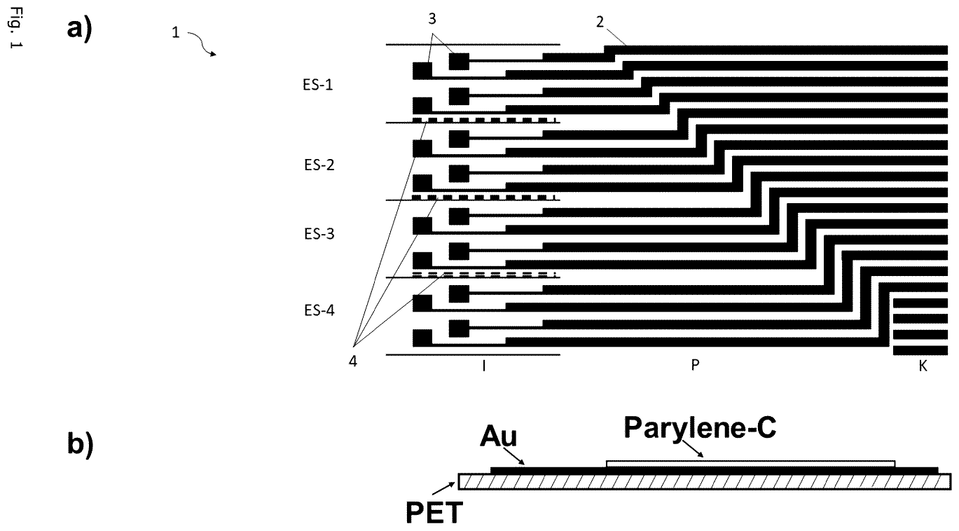

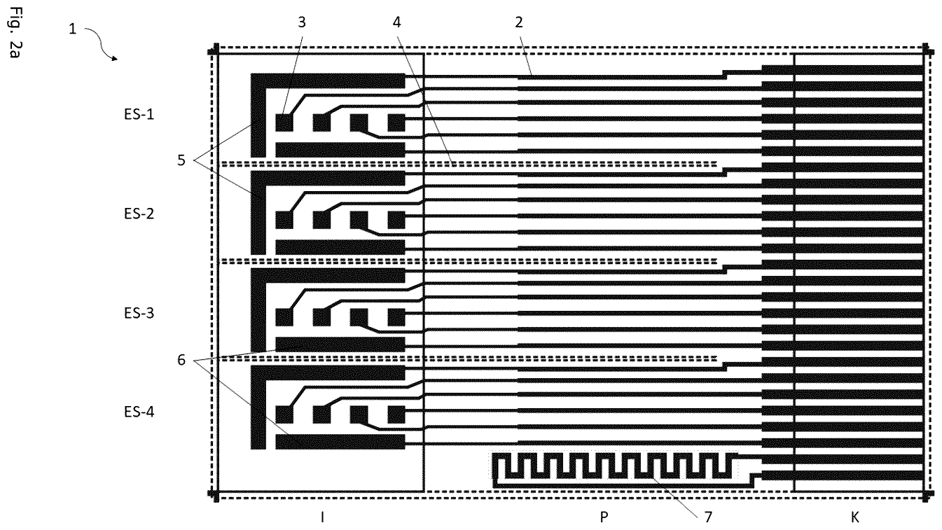

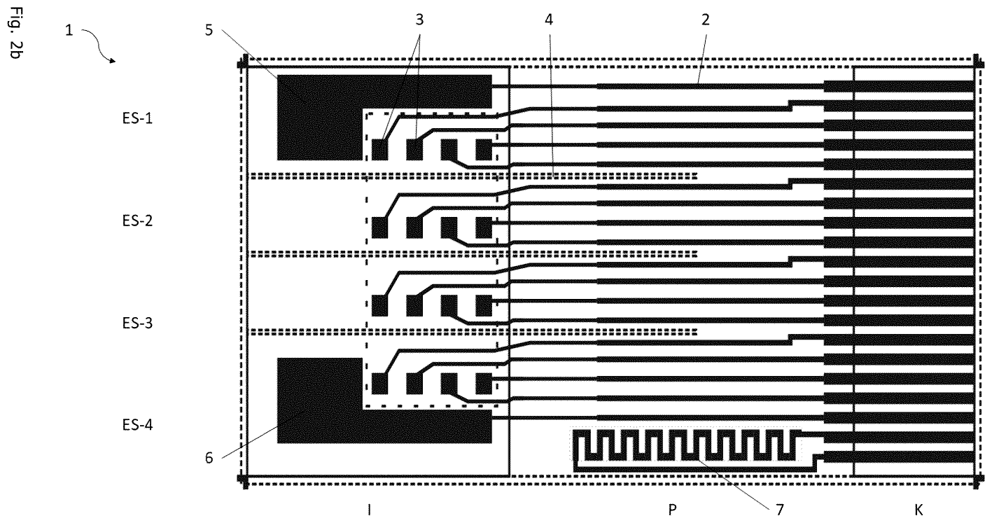

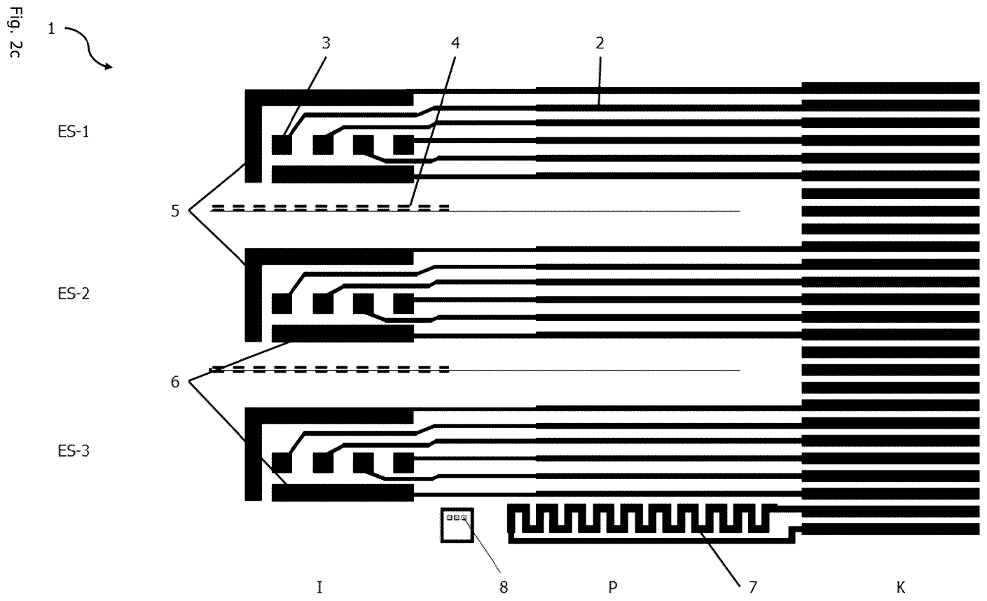

### LIST OF REFERENCE SIGNS

In the figures, the same reference signs mean the same materials, substances, etc.
1 biomarker sensor with multielectrode array structure (flex-MEA)
2 conductor path
3 aptamer-covered electrode contact/electrode contact for aptamer covering
4 electrode separation area/cutting strip
5 counter electrode
6 reference electrode
7 resistance thermometer
8 alignment marks
ES-1 electrode set 1
ES-2 electrode set 2
ES-3 electrode set 3
ES-4 electrode set 4
I incubation zone
P passivation zone
K contact zone

The present invention will now be explained in more detail with reference to the following non-limiting examples. The following non-limiting examples serve to illustrate the embodiments described therein. It will be known to the person skilled in the art that variations of these examples are possible within the scope of the present invention.

### EXAMPLES

### Example 1: Malaria

### 1. Preparation and Purification of Flexible Multielectrode Arrays Chips

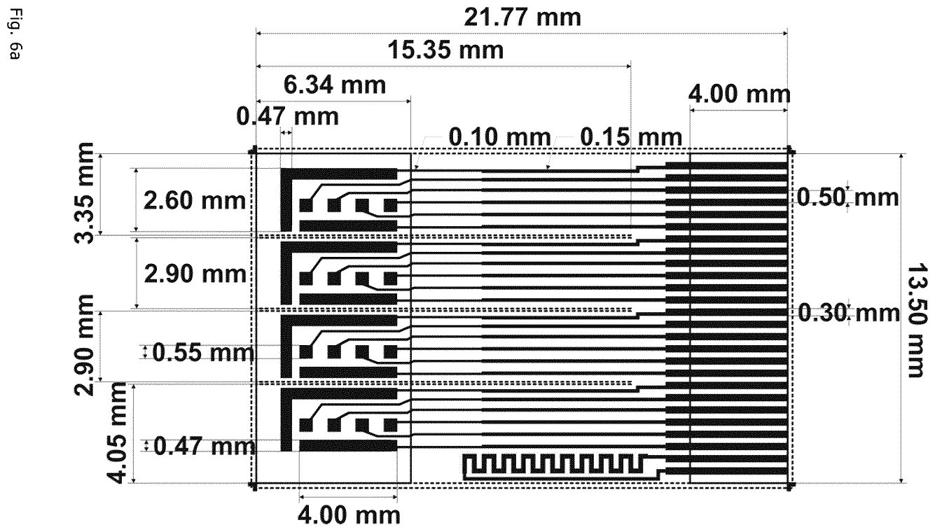

Flexible multielectrode arrays (flex-MEA) were prepared in an ISO 1-3 clean room on a polyethylene terephthalate film (PET, DuPont Teijin Films Ltd.) of a thickness of 100 μm and a diameter of 100 mm as a flexible substrate. Electrode beam assisted physical vapor deposition (PVD) was used to first deposit a 5 nm titanium adhesion layer and then thereon a layer of 50 nm gold (Au) on the flexible substrate for the preparation of the electrode. An etch mask for the patterns of the leads and the 16 electrodes (four electrodes each for four electrode sets) was prepared by standard photolithography with a positive photoresist using a mask aligner, applied to the gold surface and then the electrodes were patterned by a wet chemical etching process using a gold etchant (TechniEtch AC12, Microchemicals, Ulm, Germany). The exposed titanium layer was then removed by wet chemical etching using a titanium etchant (TechniEtch TC, Ulm, Germany). The etch mask was then removed by immersing the PET substrate comprising the patterned electrodes in AZ-100 remover in an ultrasonic bath for 10 minutes, followed by rinsing with isopropanol and deionised water. The flexible polymer chips obtained were 10.5 mm×15.8 mm in size, with 16 individually addressable electrodes (see also FIG. 5). Each square-shaped electrode with a size of 550 μm×550 μm is arranged on the so-called incubation side (or incubation zone) of the chip, whereby this incubation side of the chip serves as the base for the flex-MEA.

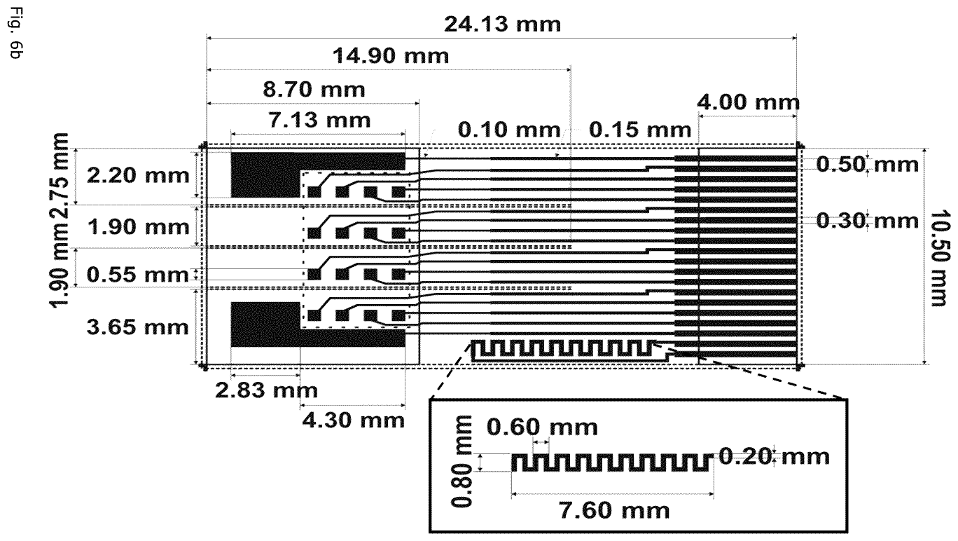

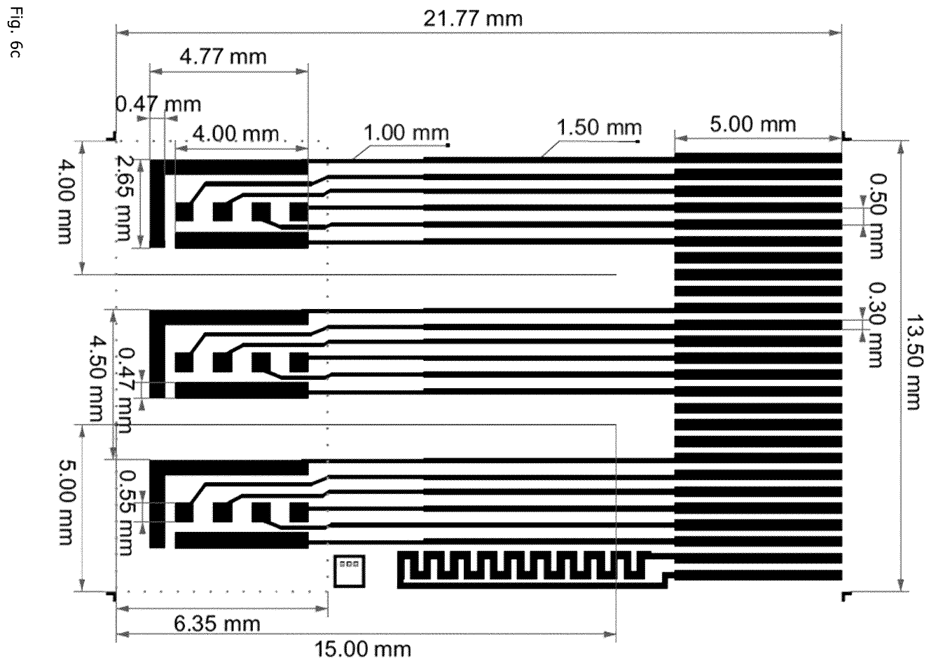

In addition, further flex-MEA designs comprising additionally an on-chip reference electrode 6 (RE), a counter electrode 5 (CE) and a resistance temperature detector 7 (RTD) have been prepared (see also FIG. 6).

The passivation layer was prepared by means of stencil passivation using parylene C to form the passivation layer (protective layer over the electrode leads). A polymer stencil was made for this using a laser cutting machine. The stencil was then applied to the PET substrate with the structured electrode. Parylene C was then deposited by chemical vapor deposition (CVD) with the following parameters: vacuum pressure 20 mTorr, Set Point 25 mTorr and Vaporizor 160° C. Finally, the stencil was carefully peeled off the PET substrate.

For final cleaning before use, the new flex-MEA chips were each immersed for 5 minutes in acetone and isopropanol, followed by a rinsing with highly purified deionised water (from a Milli-Q® system (18.2 megohm resistivity)) and drying in a nitrogen flow. The chemically cleaned electrodes were connected to a printed circuit board with a zero-force socket connection that allowed the connection to a potentiostat. Electrochemical cleaning of the flex-MEA chip was first performed by cyclic voltametry (CV) in 0.1 M NaOH in a potential range from −1.35 V to −0.35 V during 10 scans at 2 V s⁻¹ followed by scanning in 0.05 M H₂SO₄ in a potential range from 0 V to 1.5 V during 20 scans at 1 V s⁻¹. The electrochemical surface area (ESA) was determined by CV in 0.05 M H₂SO₄ in a potential range from 0 V to 1.5 V at 0.1 V s⁻¹.

### 2. Flex-MEA Multi-Target Aptasensor Biofunctionalization

The following concentrations were used for the single-stranded desoxyribonucleic acid (ssDNA) aptamers listed in Table 1: 0.5 μM for 2008s, pL1 and 2106s and 0.03 μM for LDHp11. All aptamers were incubated separately with 10 mM tris-(2-carboxyethyl)-phosphine hydrochloride (TCEP) solution for one hour at room temperature to release the disulfide protective bond and allow immobilisation on the electrodes via a thiol-gold self-assembling monolayer. The solutions were resuspended in 10 mM phosphate-buffered saline (PBS-NaCl 1 M, NaH₂PO₄ 10 mM, Na₂HPO₄ 10 mM, MgCl₂ 1 mM, pH 7.1) to a final volume of 1 mL after one hour. Four parallel strip-like sections were then cut into the flex-MEA using scissors, taking care to leave the electrodes and leads intact, which made possible the electrode incubation with the four different aptamer solutions for the final preparation of the multi-target aptasensor. The individual cut electrodes could be bent individually due to the flexible substrate, so that a respective immersion in the chosen incubation solution without risking the other electrode arrays being incubated as well was made possible. The electrodes were then incubated with their incubation zone individually overnight (16 hours) with the respective aptamer solution under exclusion of light.

TABLE 1
Sequences of the receptor aptamers used
for the multi-target flex MEA aptasensor

| Aptamer | Sequence |
|---|---|
| 2008s | 5′-HO—(CH₂)₆—S—S—(CH₂)₆—O— |
| | CTG GGC GGT AGA ACC ATA GTG |
| | ACC CAG CCG TCT AC-3′ |
| pL1 | 5′-HO—(CH₂)₆—S—S—(CH₂)₆—O— |
| | GTT CGA TTG GAT TGT GCC GGA |
| | AGT GCT GGC TCG AAC-3′ |
| LDHp11 | 5′-HO—(CH₂)₆—S—S—(CH₂)₆—O— |
| | CTA CTG TTG ATA TGA GTG ATA |
| | GGG CGG CGC GCT TAT CTG TAT |
| | TGT G-3′ |
| 2106s | 5′-HO—(CH₂)₆—S—S—(CH₂)₆—O— |
| | GCT TAT CCG ATG CAG ACC CCT |
| | TCG GTC CTG CCC TC-3′ |

### 3. Washing

The aptamer-modified flex-MEA chip was first rinsed with 25 mM Tris-HCl buffer (NaCl 0.1 M, tris(hydroxymethyl)aminomethane 25 mM, HCl 25 mM, pH 7.5) and then with deionised water (Milli-Q® water) to remove non-specifically adsorbed molecules.

### 4. Blocking

The flex-MEA chip was incubated with a 5 mg/ml monofunctional methoxy polyethylene glycolthiol solution (PEG, 2 kDa) for 7 hours. The PEG served as a blocking molecule to prevent biofouling by other molecules present in the blood samples.

### Washing

The flex-MEA aptasensor was washed with Tris buffer to remove excess not specifically adsorbed PEG molecules.

### 6. Analyte Detection

Blood samples containing the Plasmodium falciparum parasite were mixed 1:1 with lysis buffer (25 mM Tris-HCl buffer with 0.5% Triton X-100 (octylphenol ethoxylate), is used to break up the cells of the parasite). After 15 minutes incubation with the buffer, the resulting lysed parasitised blood was diluted in 25 mM Tris buffer 1:100 to a final volume of 2 ml. The aptasensor was incubated with this diluted sample for 45 minutes.

The output signals obtained from the different electrodes, which were each modified with a particular aptamer receptor, are shown in Table 2. The signal was considered as 0 or 1 if the measured current was below or above a certain threshold. The threshold was assumed to be 3σ (3-sigma), where σ (sigma) is the standard deviation of the analyte-free measurement. Both the 2008s as well as the pL1 aptamer target PfLDH and PvLDH, resulting in highly redundant signals leading to a high reliability of the malaria test. The LDHp11 and 2106s aptamers, which selectively target PfLDH and HRP-2, respectively, allow discrimination between P. falciparum and P. vivax malaria parasites.

The output significance indicates whether the combination of the respective inputs is meaningful for the diagnosis or not.

TABLE 2
Input and output table for the different biomarker detections
by sensors with respective aptamers (Inp in this and
the following tables = abbreviation of Input)

| | Inp 1 | Inp 2 | Inp 3 | | Output |
|---|---|---|---|---|---|
| Aptamer | (PfLDH) | (PvLDH) | (HRP-2) | Output | significance |
| 2008s | 0 | 0 | 0 | 0 | clear |
| | 1 | 0 | 0 | 1 | unclear |
| | 0 | 1 | 0 | 1 | clear |
| | 1 | 1 | 0 | 1 | unclear |
| | 0 | 0 | 1 | 1 | unclear |
| | 1 | 0 | 1 | 1 | clear |
| | 0 | 1 | 1 | 1 | unclear |
| | 1 | 1 | 1 | 1 | clear |
| pL1 | 0 | 0 | 0 | 0 | clear |
| | 1 | 0 | 0 | 1 | unclear |
| | 0 | 1 | 0 | 1 | clear |
| | 1 | 1 | 0 | 1 | unclear |
| | 0 | 0 | 1 | 1 | unclear |
| | 1 | 0 | 1 | 1 | clear |
| | 0 | 1 | 1 | 1 | unclear |
| | 1 | 1 | 1 | 1 | clear |
| LDHp11 | 0 | 0 | 0 | 0 | clear |
| | 1 | 0 | 0 | 1 | clear |
| | 0 | 1 | 0 | 0 | clear |
| | 1 | 1 | 0 | 1 | unclear |
| | 0 | 0 | 1 | 0 | unclear |
| | 1 | 0 | 1 | 1 | clear |
| | 0 | 1 | 1 | 0 | unclear |
| | 1 | 1 | 1 | 1 | unclear |
| 2106s | 0 | 0 | 0 | 0 | clear |
| | 1 | 0 | 0 | 0 | unclear |
| | 0 | 1 | 0 | 0 | clear |
| | 1 | 1 | 0 | 0 | unclear |
| | 0 | 0 | 1 | 1 | unclear |
| | 1 | 0 | 1 | 1 | clear |
| | 0 | 1 | 1 | 1 | unclear |
| | 1 | 1 | 1 | 1 | unclear |

The output from the respective sensors resulted in a lot of ambiguous information (e.g., due to cross-sensitivities), so that in many scenarios it was impossible to target the infections if only the output from one sensor at a time was considered.

To set the outputs to “clear” and allow a selective and unambiguous detection of the biomarker and a corresponding diagnosis, logical operations were performed involving input and output tables (Tables 3, 5 and 7, see below). Thereby the different outputs of the electrodes were combined on one chip. For this, the outputs of the respective sensors (Table 2) were used as input for logic gates and corresponding truth tables were created for specific parasitic infection scenarios that were checked with the sensor (Tables 4, 6 and 8, see further below). The combinations of the different aptamer outputs and processing of these sensor outputs by logic gate operations allowed the unambiguous detection of malaria infections, the discrimination between different malaria parasites, the confirmation of the result by redundant sensor analysis, as well as the discarding of the probability of false positive results, as in Table 8. Furthermore, all electrodes of an electrode set were modified with the same aptamer receptor, and therefore the signals coming from the different electrodes were averaged. Averaging the gathered data in a parallel manner increased the reliability of the measurement information.

In the following, three partial examples T-1, T-2 and T-3, are presented for logical operations as they were performed, but other logical operations of different levels can also be performed.

### Partial-Example T-1: Plasmodium falciparum

TABLE 3
Input and output table for sensors modified with LDHp11 aptamer
and 2106s aptamer for the detection of different combinations
of biomarkers. The output is only 1 if both Plasmodium falciparum
biomarkers (PfLDH + HRP-2) are detected. Co-infection
with Plasmodium vivax is not excluded.

| | Inp 1 | Inp 2 | Output |
|---|---|---|---|
| | LDHp11 | 2106s | P. falciparum |
| 0 | 0 | 0 | 0 |
| PfLDH | 1 | 0 | 0 |
| PvLDH | 0 | 0 | 0 |
| PfLDH + PvLDH | 1 | 0 | 0 |
| HRP-2 | 0 | 1 | 0 |
| PfLDH + HRP-2 | 1 | 1 | 1 |
| PvLDH + HRP-2 | 0 | 1 | 0 |
| PvLDH + PfLDH + HRP-2 | 1 | 1 | 1 |

TABLE 4
Truth table of the combination of LDHp11 and 2106s for
P. falciparum infections using AND gates. Logic diagram:
Output = Inp 1 × Inp 2

| Inp 1 | Inp 2 | Output |
|---|---|---|
| LDHp11 | 2106s | P. falciparum |
| 0 | 0 | 0 |
| 1 | 0 | 0 |
| 0 | 1 | 0 |
| 1 | 1 | 1 |

1: Represents an unambiguous positive response according to the addressed task (here: infection with Plasmodium falciparum).
0: Represents an equivocal test result due to cross-selectivity of the aptamer receptors or no infection.

### Partial-Example T-2: Plasmodium vivax Infection Only

TABLE 5
Input and output table for the combinations of detection of
2008s, LDHp11 and 2106s aptamers for P. vivax detection only.
A co-infection with P. falciparum is excluded.

| | Inp 1 | Inp 2 | Inp 3 | Output |
|---|---|---|---|---|
| | 2008s | LDHp11 | 2106s | only P. vivax |
| 0 | 0 | 0 | 0 | 0 |
| PfLDH | 1 | 1 | 0 | 0 |
| PvLDH | 1 | 0 | 0 | 1 |
| PfLDH + PvLDH | 1 | 1 | 0 | 0 |
| HRP-2 | 1 | 0 | 1 | 0 |
| PfLDH + HRP-2 | 1 | 1 | 1 | 0 |
| PvLDH + HRP-2 | 1 | 0 | 1 | 0 |
| PvLDH + PfLDH + HRP-2 | 1 | 1 | 1 | 0 |

TABLE 6
Truth table of the combination of 2008s, LDHp11 and 2106s only for
P. vivax infection with NOT/NOR - NOT/NOR gate: logic diagram:
output = Inp 1 + Inp 2 + Inp 3.

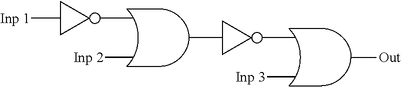

| Inp 1 | Inp 2 | | Inp 3 | Output 2 |
|---|---|---|---|---|
| 2008s | LDHp11 | Output 1 | 2106s | P. vivax |
| 0 | 0 | 0 | 0 | 0 |
| 1 | 0 | 1 | 0 | 1 |
| 0 | 1 | 0 | 0 | 0 |
| 1 | 1 | 0 | 0 | 0 |
| 0 | 0 | 0 | 1 | 0 |
| 1 | 0 | 1 | 1 | 0 |
| 0 | 1 | 0 | 1 | 0 |
| 1 | 1 | 0 | 1 | 0 |

### Partial-Example T-3: Confirmation of Detection by Redundant Sensor Signals

TABLE 7
Input and output table for the combination of the detection of 2008s
and pL1 aptamers for P. falciparum and P. vivax mixed infections
using redundant signal to reject a false positive result.

| | | | Output |
|---|---|---|---|
| | Inp 1 | Inp 2 | P. falciparum |
| | 2008s | pL1 | and P. vivax |
| 0 | 0 | 0 | 0 |
| PfLDH | 1 | 1 | 1 |
| PvLDH | 1 | 1 | 1 |
| PfLDH + PvLDH | 1 | 1 | 1 |
| HRP-2 | 1 | 1 | 1 |
| PfLDH + HRP-2 | 1 | 1 | 1 |
| PvLDH + HRP-2 | 1 | 1 | 1 |
| PvLDH + PfLDH + HRP-2 | 1 | 1 | 1 |

TABLE 8
Truth table for the combination of 2008s and pL1-modified
sensors for redundant confirmation of a P. falciparum
and P. vivax co-infection detection using AND gates.
Logic diagram: Output = Inp 1 × Inp 2

| | | Output P. falciparum |
|---|---|---|
| Inp 1 2008s | Inp 2 pL1 | and P. vivax |
| 0 | 0 | 0 |
| 1 | 0 | 0 |
| 0 | 1 | 0 |
| 1 | 1 | 1 |

### 7. Washing

The flex-MEA aptasensor was then rinsed with Tris buffer to remove unbound proteins.

### 8. Measurement of the Electrochemical Multi-Target Aptasensor Detection Signal

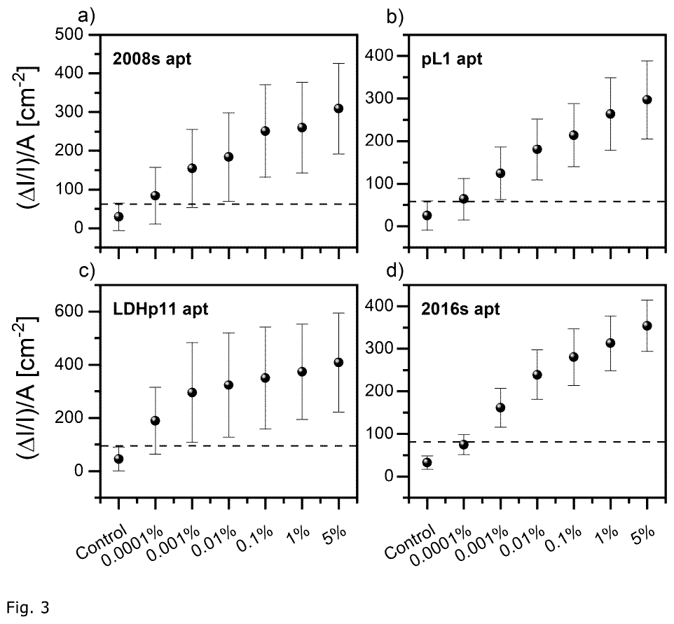

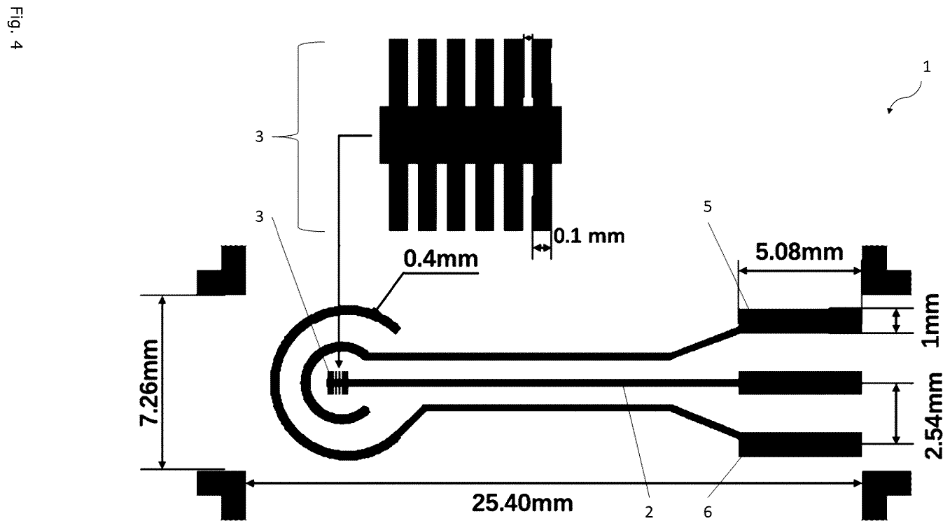

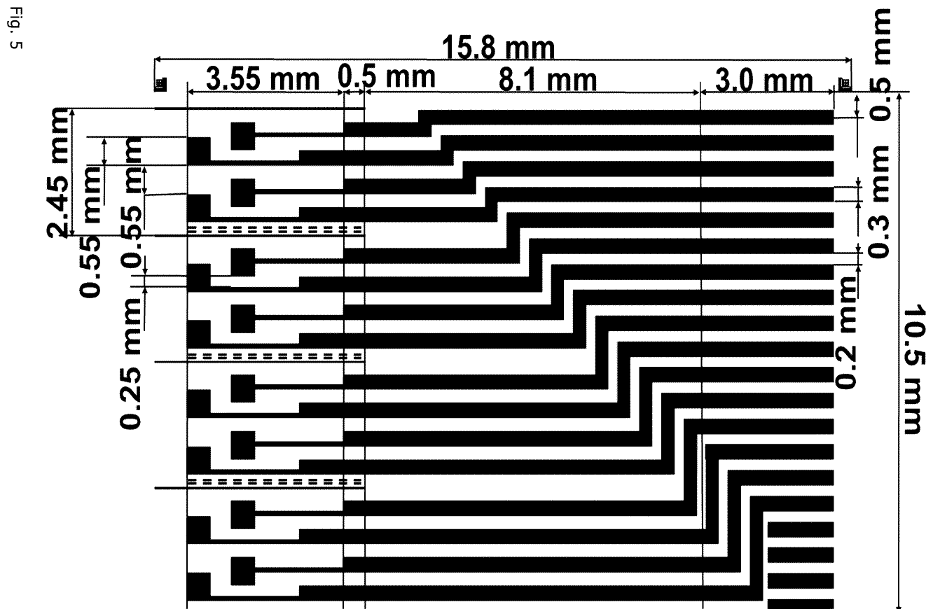

Differential pulse voltammetry (DPV) measurements were performed to determine the detection of PfLDH and HRP-2 aptasensors in the parasitised blood samples. DPV measurements were performed in 5 mM potassium ferri- and ferrocyanide ([Fe(CN)₆]^{3-/4-}) in 25 mM Tris buffer solution using a multichannel potentiostat with a three-electrode system. For this a platinum wire was used as counter electrode (CE), an Ag/AgCl electrode as reference electrode (RE) and the flex-MEA multi-target aptasensor as working electrode (WE). The DPV measurements were performed in a potential range of 0 V to 0.7 V, with a step potential of 0.005 V, an amplitude modulation of 0.025 V, an equilibrium time of 2 seconds, a pulse width of 0.05 seconds and a measurement width of 0.025 seconds. The statistical analysis of the detection with the different electrodes and the different aptamers was carried out and evaluated using statistical analysis software (see also FIG. 3).

With the biomarker sensors according to the invention, malaria infections could be reliably detected in blood samples with very high accuracy.

### Example 2: SARS-CoV-2

In this example, the flexMEA sensors according to the invention were used for the detection of the spike protein of the SARS-CoV-2 virus. For this purpose, the C7 aptamer, which binds to the spike protein of the SARS-CoV-2 virus, was used as the aptamer.

The aptasensor was incubated with the spike protein for 30 minutes and then rinsed with Tris buffer (as described above). The electrochemical signal detection was performed with a potentiostat using differential pulse voltametry (DPV), wherein the DPV measurements took place particularly in redox species solution (5 mM [Fe(CN)₆]^{3-/4-} in 25 mM Tris buffer).

After normalizing the peak currents which were obtained in the detection of the spike protein at different concentrations, a calibration curve corresponding to the C7 aptamer was obtained. It was possible to determine a detection limit of 100 ag/ml, a sensitivity of 8.8+0.9/decade and a dynamic detection range covering concentrations from 100 ag/ml to 100 ng/ml.

Accordingly, it was shown that this electrochemical biosensor showed an outstanding performance, and even exceeded the detection limits which were reported for other methods, for example for rapid (lateral flow) assays.

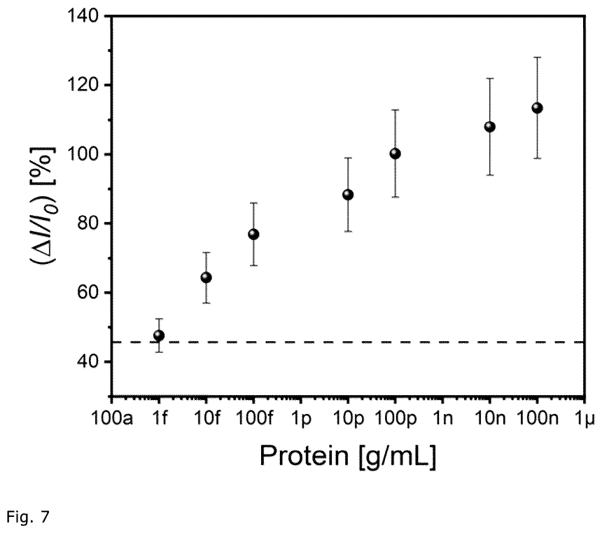

The sensor data obtained showed that the sensors according to the invention are excellently suited to detect the spike protein of the SARS-CoV-2 virus unambiguously and at low concentrations (see also FIG. 7).

The following findings were obtained from/in the experiments carried out, i.e. corresponding work was carried out, but are formulated in the present tense due to their validity for future work.

In a multi-target determination, the flex-MEA sensor of the present invention can be used as a logic gate. Accordingly, the detected signals from each electrode of the biosensor serve as input signals (inputs) to the logic gate, the output of which corresponds to the result of the Boolean operation, whereby the positive (1) or negative (0) identification of the pathogen is indicated, or whereby the presence + positive (1) or absence − negative (0) of the pathogen is indicated. Such a concept is illustrated in the following tables C-A and C-B with an example of a multitarget determination of different spike protein variants. Here, the biosensors function as exclusive-or gates (XOR gates) to distinguish between COVID-19 and its different variants, the spike protein and SARS-CoV-2 virus are the inputs to the gate. These logic gates facilitate an unambiguous determination of the infections with the possibility to distinguish between the different infecting viruses or to validate a possible co-infection with other pathogens, or to discard the possibility of a false positive result by averaging over several redundant sensor signals from the different electrodes.

Table C-A shows the three different aptamers and their corresponding spike targets. When the target is exposed to its aptamer receptor (InpX=1), the output receives status 1. When the target is not present (InpX=0), the output receives status 0. Considering all input combinations, multiple spike proteins may also be present, but this does not necessarily reflect a realistic situation that may be found in real samples. In this respect, the output significance indicates whether the combination of the different target inputs reflects a meaningful biomarker combination that clearly indicates an infection or no infection. In this case, the result is “clear”. Random combinations are taken as “unclear”. The presence of two different spike proteins which represent a co-infection (unlikely) are taken as unclear.

TABLE C-A
Example of a discriminatory detection of a COVID-19
infection between different virus variants.

| | | Inp 2 | Inp 3 | | |
|---|---|---|---|---|---|
| | Inp 1 | (spike | (spike | | |
| | (spike | protein | protein | | Output |
| Aptamer | protein) | alpha) | delta) | Output | significance |
| C9 (alpha | 0 | 0 | 0 | 0 | clear |
| variant) | 1 | 0 | 0 | 0 | clear |
| | 0 | 1 | 0 | 1 | clear |
| | 1 | 1 | 0 | 1 | unclear |
| | 0 | 0 | 1 | 0 | clear |
| | 1 | 0 | 1 | 0 | clear |
| | 0 | 1 | 1 | 1 | unclear |
| | 1 | 1 | 1 | 1 | unclear |
| C7 (all | 0 | 0 | 0 | 0 | clear |
| variants of | 1 | 0 | 0 | 1 | clear |
| COVID-19) | 0 | 1 | 0 | 1 | clear |
| | 1 | 1 | 0 | 1 | unclear |
| | 0 | 0 | 1 | 1 | clear |
| | 1 | 0 | 1 | 1 | unclear |
| | 0 | 1 | 1 | 1 | unclear |
| | 1 | 1 | 1 | 1 | unclear |
| C5 (delta | 0 | 0 | 0 | 0 | clear |
| variant) | 1 | 0 | 0 | 0 | clear |
| | 0 | 1 | 0 | 0 | clear |
| | 1 | 1 | 0 | 0 | clear |
| | 0 | 0 | 1 | 1 | clear |
| | 1 | 0 | 1 | 1 | unclear |
| | 0 | 1 | 1 | 1 | unclear |
| | 1 | 1 | 1 | 1 | unclear |

In table C-B an example of a biosensor implementation from Table C-A, wherein the electrodes are used as XOR gates to discriminate between different variants of COVID-19 is shown. The different spike proteins of the SARS-CoV-2 variants are used as input signals to the gate. These logic gate facilitates an unambiguous determination of infections with the ability to distinguish between the different infecting viruses and rule out co-infections by processing the different redundant sensor signals coming from the different electrodes and discarding the possibility of a false positive output. Various other logic gates can be created from the possible combinations of aptamer-based biosensor signals coming from the same flexible chip and used for specific disease discrimination.

TABLE C-B
XOR logic gate inputs and outputs for two sets of sensors which were
modified differently with either C9 or C5 aptamers for the detection
of different COVID-19 biomarkers. The output is only 1 if one of
the biomarkers is detected. A co-infection is excluded just as an
infection with the wild type. Output 1 is only obtained if the aptasensors
give a signal above their respective detection threshold.

| | Inp 1 C9 | Inp 3 C5 | Output COVID-19 |
|---|---|---|---|
| 0 | 0 | 0 | 0 |
| spike protein | 0 | 0 | 0 |
| spike protein alpha | 1 | 0 | 1 |
| spike protein delta | 0 | 1 | 1 |
| spike protein alpha + | 1 | 1 | 0 |
| spike protein delta | | | |

TABLE C-C
Truth table of an XOR logic gate, corresponding to Table C-B. Input
1 (Inp 1) is spike protein alpha and input 2 (Inp 2) is spike
protein delta. Outputs for two sets of differently modified sensors,
with either C9 or C5 aptamers, for the detection of different
COVID-19 biomarkers. Logic diagram: Output = Inp1 ⊕ Inp2

| Inp 1 C9 | Inp 2 C5 | Output COVID-19 |
|---|---|---|
| 0 | 0 | 0 |
| 0 | 1 | 1 |
| 1 | 0 | 1 |
| 1 | 1 | 0 |

1 indicates an umambiguous positive response to the task in question (here infection with Covid-19 alpha or Covid-19 delta).
0 indicates an unclear test result due to cross-selectivity of the aptamer receptors or no infection.

Such tables have accordingly been generated with other aptamers, in particular also with the C15 aptamer (which is relevant for the omicron variant of SARS-CoV2); but not shown again here due to “sameness”.

In another embodiment, different sets of sensors of the same flexible multielectrode chip have been modified with either C7 aptamer, which detects the spike protein of the SARS-CoV-2 virus, or with I1 aptamer, which detects the HA1 protein of the influenza virus, to specifically detect COVID-19 infections. The output is only 1 if the spike protein is detected. A 1 was obtained as output only when the C7 aptasensor gives a signal above its respective threshold, which is set by the detection limit threshold of the biosensor.

Corresponding sensors have also already been produced in which aptamers, namely the I1 aptamer, the SG1 aptamer, the C15 aptamer, were used which encompass further biomarkers: Haemagglutinin (HA1) protein of the influenza virus, the glycoprotein G of the HRS virus and the spike protein of the MERS-CoV and in particular also the Omicron variant of the SARS-CoV-2 virus.

1.-15. (canceled)

16. A biomarker sensor, wherein the sensor has a multielectrode array structure which comprises a carrier substrate on which at least two separate electrode sets are arranged, each electrode set comprising one or more electrodes, and each electrode set being divided into, in the following order
(i) an incubation zone in which at least one specific aptamer is bound to the one or more electrodes,
(ii) a passivation zone in which a passivation layer covers the one or more electrodes,
(iii) a contact zone in which the one or more electrodes are configured to be electrically contacted,
incubation zones of individual electrode sets or incubation zones and a part of up to 95% of a length of passivation zones of the individual electrode sets being configured to be movable, in each case independently of those of the other electrode sets, and contact zones of the individual electrode sets together forming a common contact zone.

17. The biomarker sensor of claim 16, wherein the carrier substrate is a material selected from polyethylene terephthalate, polyethylene naphthalate, polydimethylsiloxane, polyimide, polyester, and agarose.

18. The biomarker sensor of claim 16, wherein the carrier substrate is a flexible material.

19. The biomarker sensor of claim 16, wherein the sensor comprises two, three or four separate electrode sets.

20. The biomarker sensor of claim 19, wherein the sensor sets comprise at least two electrodes each.

21. The biomarker sensor of claim 19, wherein the sensor sets comprise at least four electrodes each.

22. The biomarker sensor of claim 16, wherein the electrodes are made of noble metal or carbon.

23. The biomarker sensor of claim 16, wherein the at least one specific aptamer is selected from one or more of 2008s aptamer, 2106s aptamer, pL1 aptamer, and LDHp11 aptamer.

24. The biomarker sensor of claim 16, wherein the at least one specific aptamer is selected from one or more of C5 aptamer, C7 aptamer, C9 aptamer, C11 aptamer, C15 aptamer or I1 aptamer, IH1 aptamer, SG1 aptamer, HCS1 aptamer, and NG1 aptamer.

25. The biomarker sensor of claim 16, wherein different aptamers or aptamer mixtures are bound in each electrode set.

26. The biomarker sensor of claim 25, wherein the different aptamers or aptamer mixtures bound in each electrode set are (1) in a first electrode set 2008s aptamer and in a second electrode set 2106s aptamer and in a third electrode set pL1 aptamer and in a fourth electrode set LDHp11 aptamer or (2) in a first electrode set C7 aptamer and in a second electrode set CI1 aptamer or (3) in at least two electrode sets aptamers selected from one or more of C5 aptamer, C7 aptamer, C9 aptamer, C11 aptamer, C15 aptamer or I1 aptamer, IH1 aptamer, SG1 aptamer, HCS1 aptamer, and NG1 aptamer.

27. The biomarker sensor of claim 16, wherein the sensor further comprises:
(i) collectively a reference electrode and a counter electrode,
(ii) collectively one reference electrode, one counter electrode, and one resistance thermometer,
(iii) one reference electrode and one counter electrode per electrode set, or
(iv) one reference electrode and one counter electrode per electrode set and collectively one resistance thermometer.

28. A method for producing a biomarker sensor, wherein the method comprises
(I) providing a carrier substrate,
(II) applying a layer of electrode material to the carrier substrate,
(III) structuring the electrode material resulting from (II) into individual electrodes,
(IV) passivating a part of the electrode material located between ends thereof,
(V) dividing the electrodes into a number of individual electrode sets,
(VI) incubating one end of each electrode set in aptamer solution.

29. The method of claim 28, wherein application of a layer of the electrode material to the carrier substrate is carried out by physical vapor deposition.

30. The method of claim 28, wherein in (III) first a photoresist is applied to the electrode material, a mask with a desired patterning of metal structure is produced from the photoresist by exposure to light and development, unneeded material of an adhesion layer and of the electrode layer is subsequently removed by means of wet chemical etching, and thereafter the photoresist is removed again.

31. The method of claim 28, wherein the passivation in (IV) is carried out by stencil passivation, wherein first a stencil is placed on a surface of the electrodes, and then conductive leads of the electrodes are partially passivated by chemical vapor deposition.

32. The method of claim 28, wherein a subdivision in (V) is performed by cutting the carrier substrate in such a way that a total number of electrodes is evenly distributed among the resulting electrode sets.

33. A method for the determination of biomarkers in a body fluid, wherein the method comprises
(i) providing the biomarker sensor of claim 16,
(ii) applying a body fluid sample to the biomarker sensor,
(iii) connecting the biomarker sensor to a measurement device for current voltage, resistance or frequency,
(iv) detecting a measurement signal and outputting measurement values by the measurement device,
(v) analyzing the output values, and
(vi) optionally storing, displaying and/or transmitting analyzed results.

34. A biomarker sensor kit, wherein the kit comprises or consists of
(A) at least one biomarker sensor according claim 16 without aptamers already being bound,
(B) a preparation comprising at least one biomarker-specific aptamer,
(C) optionally further analysis materials.

35. A method for the qualitative or quantitative determination of a disease-specific biomarker in a body fluid sample, wherein the method comprises using the biomarker sensor of claim 16 for determining the disease-specific biomarker.
